(19) United States
(12) Patent Application Publication
(10) Pub. No.: US 2017/0216175 A1
(43) Pub. Date: Aug. 3, 2017

(54) USE OF BIS-AMINO COMPOUNDS FOR TREATMENT OF HAIR

(71) Applicant: Active Concepts, LLC, Lincolnton, NC (US)

(72) Inventors: Durant Scholz, Nanuet, NY (US); Erica Segura, Huntersville, NC (US)

(21) Appl. No.: 15/421,856

(22) Filed: Feb. 1, 2017

Related U.S. Application Data
(60) Provisional application No. 62/289,493, filed on Feb. 1, 2016.

(51) Int. Cl. A61K 8/44; A61K 8/64; A61Q 5/06; A61K 8/73; A61Q 5/08; A61Q 5/00

(52) U.S. Cl. CPC A61K 8/44; A61Q 5/08; A61Q 5/004; A61Q 5/00; A61Q 5/065; A61K 8/73; A61K 8/64; A61K 2800/596

(57) ABSTRACT

A method for the treatment of hair that includes the following steps: providing a pretreatment solution that includes a bis amino compound having a α-Hydroxy acid attached thereto; causing the pretreatment solution to react such that polymeric chains are formed; forming the polymeric chains into a biofilm; exposing the mixture to at least one hair; and wherein the biofilm defines a lattice structure.

### BACKGROUND OF THE INVENTION

The present invention relates to a bis-amino compound and more specifically to bis-amino compounds and a method of using them for treatment of hair.

### BRIEF DESCRIPTION OF THE INVENTION

There are many different treatment applications that are made to hair. Hair can be treated to be colored, hair can be treated to be bleached, straight hair can be treated to be made curly, and curly hair can be treated to be made straight. Many, if not all, of these treatments can cause post-treatment damage.

Such damage is often caused by hair bleaching and/or dying. These treatments can be prone to leave hair stripped, dry, and cracked. As a result of treatments, the hair can have a “burnt” appearance. In addition, after bleaching or dying hair is often weaker and significantly more prone to breakage. In other words, hair after bleaching and dying has a lower tensile strength, or ultimate strength, relative to untreated hair. The total stress a material can withstand while being stretched and pulled before failing a breaking is referred to as tensile strength.

Thus there is a need for a product to reduce or eliminate damage caused to hair by treatments such as bleaching and dying. There is also a need for a pretreatment configured to reduce or eliminate damage caused by products configured to straighten or soften coarse, curly, kinky, ethnic, and/or frizzy hair.

### SUMMARY OF THE INVENTION

The present invention addresses this problem by providing a method for the use of a bis amino compound having a α-Hydroxy acid attached thereto as a pretreatment solution for hair.

According to one aspect of the present invention, there is provided a method for the treatment of hair that includes the following steps: providing a pretreatment solution that includes a bis amino compound having a α-Hydroxy acid attached thereto; causing the pretreatment solution to react such that polymeric chains are formed; forming the polymeric chains into a biofilm; exposing the mixture to at least one hair; and wherein the biofilm defines a lattice structure.

### BRIEF DESCRIPTION OF THE DRAWINGS

The invention may be best understood by reference to the following description taken in conjunction with the accompanying drawing figures in which:

FIG. 1 shows a standard electron microscopy image of untreated hair;

FIG. 2 shows a standard electron microscopy image of bleached hair;

FIG. 3 shows a standard electron microscopy image of hair treated according to the present invention; and

FIG. 4 shows a chart comparing untreated hair and hair treated according to the present invention with regards to how tensile strength of the hair is affected by bleaching.

### DETAILED DESCRIPTION OF THE INVENTION

The present invention provides compounds for pretreating hair such that subsequent treatments related to bleaching, dying, straightening, relaxing, curling, and the like do not cause as much damage as would be expected if the hair had not been pretreated with the compound of the present invention.

The compounds of the present invention include a bis amino compound having any α-Hydroxy acid (“AHA”) attached thereto. It should be appreciated that the AHA can be natural or formed synthetically. The present invention also includes polyamine compounds of a molecular weight range from about 80 DA to about 200 K DA and any AHA, natural or synthetic and hair care application specifically related to damaging treatments like color-treating, bleaching, relaxing, and the like. Included in this class of compounds for example are bis-amino compounds having lactic acid as a functional group.

The compounds of the present invention form composite particles configured to ionically bind to hair cuticles such that the particles of the present invention form a protective film or web around the hair cuticles. The protective film is configured to protect hair from harsh hair bleaches, free radicals, and peroxides.

The proteins of the present invention act as natural buffers to remove hydrogen radicals and to balance pH. Amino acids, such as those common in Pisum sativum can release hydrogen ions in alkaline environments thus raising pH. In acidic environments amino acids bind to excess hydrogen thereby lowering pH. In this manner, amino acids can function to buffer a solution's acidity or alkalinity.

Biofilms formed by the compounds of the present invention are polymeric chains formed from a conglomeration of proteins, amino acids, and polysaccharides. The polymeric chain is a complex matrix of molecules. It is believed that the proteins, amino acids, and polysaccharides of the compound of the present invention operate together to kickstart complex metabolic functions that operate on small-scale biochemical interactions.

The biofilm of the present invention acts as a scaffolding. The scaffolding functions to support and protect hair, but still allows small molecules and hydrogen ion to access the pretreated hair because it is semipermeable. Thus it functions In this regard, the biofilm of the present invention does not act as a true barrier. It is believed that these features allow for the biofilm formed from the compounds of the present invention to exhibit properties such as Morse recession, pH balance, barrier protection, and protection from hair weakening caused by bleaches and dyes. The protection against hair weakening is believed to be derived from the scaffolding aspect of biofilms of the present invention.

It is believed that the compounds of the present invention are operable to protect hair and still allow for reduction in pigmentation through bleaching at the same time. It is believed that compounds of the present invention operate by utilizing matrix style scaffolding and poly-compound reactions to form a self-supporting structure. The structure functions to provide protection for hair and still provide for successful and attractive hair bleaching.

The present invention can be better understood from a description thereof The present invention provides a method for the treatment of hair prior to subsequent treatments, i.e. pretreatment, such that subsequent treatments of dyeing, bleaching, or the like will not harm the hair. The method includes the following steps: A) providing a pretreatment solution that includes a bis amino compound having a α-Hydroxy acid attached thereto; B) causing the pretreatment solution that includes one of the following: proteins, amino acids, polysaccharides, and a mixture thereof

to react such that polymeric chains are formed; C) forming the polymeric chains into a biofilm; and D) exposing the mixture to at least one hair. The biofilm defines a lattice structure as described above.

The lattice structure is positioned such that it is closely associated with a surface of the hair such that the lattice structure defines a scaffolding generally surrounding the hair. Thus the hair with generally surrounding scaffolding defines pretreated hair that is supported with the scaffolding.

In another step the pretreated hair is exposed to a treatment solution that includes multiple chemical components. Such chemical components can include a bleach or a dye. The treatment solution is exposed to the lattice and some passes through. Some molecules are prevented from passing through the lattice.

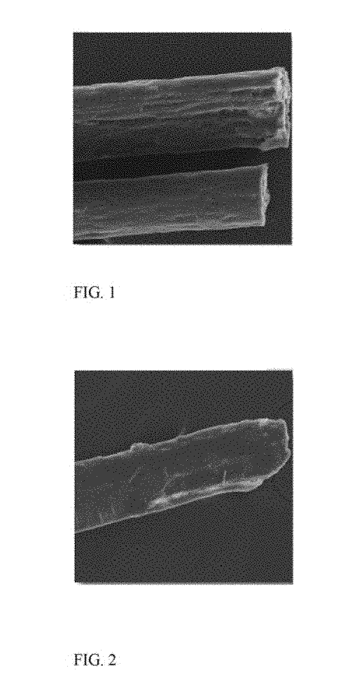

Referring now to FIG. 1, strands of hair are shown in a standard electron microscopy image. It should be noted that this untreated or virgin hair is prone to damage from everyday stresses and, as shown in FIG. 1, can exhibit characteristic signs of breakdown. Referring now to FIG. 2, hair that has not been pretreated with compounds of the present invention but has been bleached is shown. The bleached hair seen in FIG. 2 exhibits more damage of the hair fiber than that shown in FIG. 1. The additional damage of hair fiber can lead to irregular growth, breakages and overall unhealthy or dead appearance of the bleached hair. At a single level, one cuticle may not seem important, but the stranding and imperfections contribute to a much larger picture of unhealthy and unprotected hair.

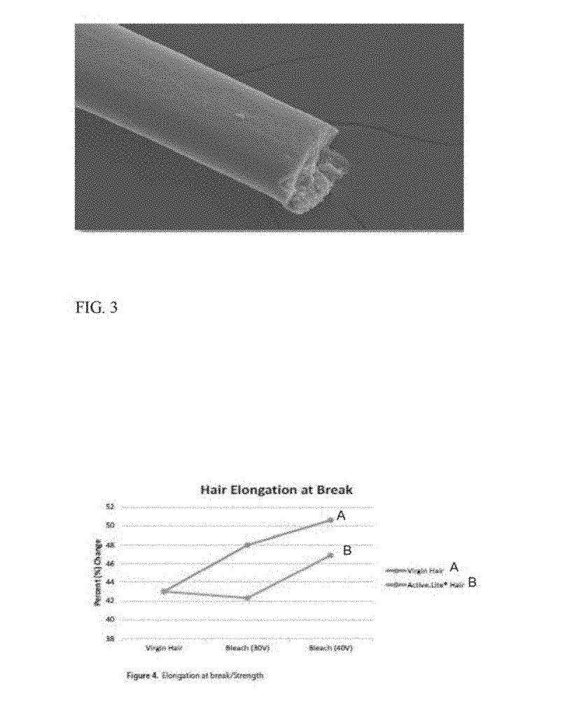

Referring now to FIG. 3, a cuticle first pretreated with a compound of the present invention, and then bleached is shown. The compounds of the present invention protect each fiber by protecting the cuticle. Leaving it smooth and annealed contributes to overall healthier looking and healthier feeling hair. The effect of the compounds of the present invention can be seen clearly when the image of FIG. 2 is compared to the image in FIG. 3. The cuticle shown in FIG. 2 exhibits more damage than the cuticle shown in FIG. 3. Should be appreciated that the combined effects of many thousands of cuticles that appear as the cuticle in FIG. 2 does would appear to be more unhealthy and unprotected than that shown in FIG. 3.

In addition to improving the appearance of hair, pretreating with compounds of the present invention allows for hair to be stronger after bleaching than it would otherwise be. Referring now to FIG. 4, and Instron device for measuring mechanical properties was used to measure the absolute strength of individually treated hair fibers. The standard procedure provided by ASTM D5034 was followed. The charts represent change in length of hair for three different conditions: untreated, bleach (30 V) and bleach (40 V). In FIG. 4, line A represents untreated, virgin hair. Line B represents hair that has been pretreated with compounds of the present invention. The pretreatment of hair represented by line B occurred prior to bleaching such that hair is protected from harmful effects caused by bleaching.

One of the benefits of the compounds of the present invention is that these compounds allow hair to exhibit both visual and sensorial properties that are most commonly associated with healthy vibrant hair when compared to hair that is been bleached in the conventional manner and not pretreated with compounds of the present invention. It should be noted that the concentration of bleach used following pretreatment of hair with compounds of the present invention can be the same concentration used when treating un-pretreated hair. Thus when compounds of the present invention are used to pretreat hair prior to bleaching the desired color can be obtained in the manner such color was obtained without pretreating with the compounds of the present.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying potential points of novelty, abstract and drawings), or to any novel one, or any novel combination, of the steps of any method or process so disclosed.

What is claimed is:

1. A method for the treatment of hair, the method comprising the steps of:
providing a pretreatment solution that includes a bis amino compound having a α-Hydroxy acid attached thereto;
causing the pretreatment solution to react such that polymeric chains are formed;
forming the polymeric chains into a biofilm;
exposing the mixture to at least one hair; and
wherein the biofilm defines a lattice structure.

2. The method for the treatment of hair according to claim 1, further comprising the step of:
positioning the lattice structure such that the lattice structure is closely associated with a surface of the hair such that the lattice structure defines a scaffolding generally surrounding the hair; and
wherein the hair with generally surrounding scaffolding defines pretreated hair.

3. The method for the treatment of hair according to claim 2, further comprising the step of:
supporting the hair with the scaffolding.

4. The method for the treatment of hair according to claim 2, further comprising the step of:
exposing the pretreated hair to a treatment solution that includes multiple chemical components.

5. The method for the treatment of hair according to claim 4, wherein the treatment solution includes a bleach.

6. The method for the treatment of hair according to claim 4, wherein the treatment solution includes a dye.

7. The method for the treatment of hair according to claim 4, further comprising the step of:
passing at least a portion of the treatment solution through the lattice.

8. The method for the treatment of hair according to claim 7, further comprising the step of preventing at least a portion of the treatment solution form passing through the lattice.

9. The method for the pretreatment of hair according to claim 4, wherein the pretreatment solution includes one of the following: proteins, amino acids, polysaccharides, and a mixture thereof.

10. The method for the pretreatment of hair according to claim 1, wherein the pretreatment solution includes a mixture of proteins, amino acids, and polysaccharides.

11. A method of pretreating hair, the method comprising the steps of:
providing a pretreatment solution that includes bis amino compound having a α-Hydroxy acid compound attached thereto;
exposing a plurality of strands of hair to the pretreatment solution;
forming a matrix style scaffolding around at least one of the plurality of strands; and
wherein the scaffolding is configured to act as a barrier to chemical transfer.

12. A method of pretreating hair according to claim 11, further comprising the step of supporting at least one of the plurality of strands with the scaffolding.

13. A method of pretreating hair according to claim 11, further comprising the step of allowing pigmentation reduction.

14. A method of pretreating hair according to claim 11, further comprising the step of balancing pH.

15. A method of pretreating hair according to claim 11, wherein the pretreatment solution includes a mixture of proteins, amino acids, and polysaccharides.
